(19) United States
(12) Patent Application Publication
(10) Pub. No.: US 2020/0138843 A1
(43) Pub. Date: May 7, 2020

(54) COMPOSITIONS FOR TREATING PAIN AND INFLAMMATION

(71) Applicant: ATLAS OLIVE OILS Sarl., Casablanca (MA)

(72) Inventor: Othmane AQALLAL, Casablanca (MA)

(21) Appl. No.: 16/676,016

(22) Filed: Nov. 6, 2019

Related U.S. Application Data
(60) Provisional application No. 62/756,169, filed on Nov. 6, 2018.

(51) Int. Cl. A61K 31/7048; A61K 31/047; A61K 31/05; A61K 31/192; A61K 31/22; A61K 9/00

(52) U.S. Cl. CPC A61K 31/7048; A61K 31/047; A61K 9/0053; A61K 31/192; A61K 31/22; A61K 31/05

(57) ABSTRACT

A pharmaceutical composition comprising antioxidants and phenolic compounds. The pharmaceutical composition includes oleuropein, hydroxytyrosol (3′4′-DHPEA) and tyrosol (p-DHPEA) and flavomoids, such as, without limiting, rutin, quercetin, luteolin and apigenin. The pharmaceutical composition further includes caffeic acid. The pharmaceutical composition further includes oleocanthal (p-DHPEA-EDA). The pharmaceutical composition wherein oleuropein is present from about at least 30%, hydroxytyrosol from about at least 50%, tyrosol from at least about 25%, and oleocanthol from at least about 2%, and suitable pharmaceutical excipients.

### CROSS-REFERENCE TO RELATED APPLICATION

The present application claims priority to U.S. Provisional Application No. 62/756,169, filed on Nov. 6, 2018, which is hereby incorporated by reference in its entirety. The documents incorporated in their entirety within the provisional application are also hereby incorporated by reference in their entirety.

### BACKGROUND

Inflammatory disease responsible for joint destruction that contributes to functional impairment. For example, rheumatoid arthritis (RA) remains the most common joint illness, occurring in 0.5-1% of worldwide population. Several factors are involved to triggering the disease: tobacco, microbiome, hormonal factors, genetic background and environmental factors. Schematically, the physiopathology of RA can be divided into three distinct phases: (1) initiation phase, (2) inflammation of the synovial membrane (synovitis), and (3) joint destruction due to the pseudotumoral proliferation of synovial cells under cytokine actions. In fact, chronic synovial inflammation is the hallmark of RA that involves complex interactions between T and B lymphocytes, macrophages, and fibroblast-like synoviocytes, including a network of cytokines, chemokines and others molecules. In RA, there is an imbalance between pro and anti-inflammatory cytokines. For instance, Nuclear Factor kappa β (NF-κβ) is activated in the synovium inflammatory cells and induced cytokines expression, including tumor necrosis factor-alpha (TNF-α), interleukine-1 (IL-1β), IL-6, IL-15, IL-18, but also metalloproteinase (MMP-1) and small osteochondral destruction molecules like prostaglandin E2 (PGE2) and nitric oxide. These inflammatory biomarkers are present with high concentrations in the synovial fluid and serum of patients with RA, which clinically manifests in swelling, pain and tissue destruction. Through the last few years, blockage of cytokines network has taken a substantial proportion in clinical management of RA, more interestedly inhibition of produced TNF-α, IL-6, and IL-1. Useful molecules blocking these cytokines are mainly represented by the monoclonal antibodies or recombinant proteins (e.g., infliximab, Etanercept). Although their simple clinical use, these substances had some unexpected effects (including, efficacy, toxicity and even pharmacodynamics), e.g., catastrophic effects of the first-into-human administration of TGN1412.

### DETAILED DESCRIPTION OF THE INVENTION

The present invention describes a composition comprising hydroxytyrosol, tyrosol (p-HPEA) and oleuropein (3,4-DHPEA-EA) that exert in vitro inhibitor effects on PGE2, LTB4, TNF-α, IL-6, IL-1 and high-sensitivity C-reactive protein (hs-CRP).

The composition described in the present invention comprises high amounts of antioxidants and phenolic compounds. In certain embodiments, the composition comprises oleuropein, hydroxytyrosol (3′4′-DHPEA) and tyrosol (p-DHPEA) along with flavomoids such as, without limiting, rutin, quercetin, luteolin and apigenin. In some embodiments, the composition comprises caffeic acid. In some embodiments, the composition further comprises oleocanthal (p-DHPEA-EDA).

In preferred embodiments of the present invention, the composition comprises at least about Oleoropein from at least about 30% to about 90%, hydroxytyrosol from about at least 50%, tyrosol from at least about 25%-90%, and oleocanthol from at least about 2% to 99%. All ranges and sub-ranges are considered to be within the disclosure of the present invention.

The present invention further discloses a composition comprising high amounts of antioxidants and phenolic compounds having suitable pharmaceutical excipients for administration to a human. All pharmaceutical excipients within the art are considered to be within the scope of the invention.

Further embodiments of the invention include dosage combinations comprising the composition of the present invention and at least one immune modulator. In a particular embodiment of the invention, the at least one immune modulator are present in a single unit dose combination. Suitable immune modulators include, but are not limited to, azathioprine, cyclosporine, tacrolimus, methotrexate, mycophenolate mofetil, and combinations thereof.

In another embodiment, the composition described herein is formulated as solid solution or a liquid solution. In preferred embodiments, the solid solution is in form a powder.

In another embodiment the present composition is combined with an insoluble, swell-able polymer, such as, but not limited to, gel-forming polymers such as crosslinked PVP, crosscarmellose sodium, and sodium starch glycolate. In certain embodiments, the dosage form may include suitable excipients, such as, but not limited to, dispersing agents.

The present invention also describes a composition for treating heart disease, coronary heart disease, inflammation, rheumatoid arthritis, tumors, cancers, diabetics cardiovascular diseases, pain, infectious disease including bacterial and fungal diseases, and anti-proliferative diseases. without cirrhosis.

The present invention includes treating inflammatory conditions by administering to a patient in need thereof a solid or liquid pharmaceutical composition of the present invention. Inflammatory conditions according to the present invention include, but are not limited to, inflammation of the esophagus, inflammation of the glottis, inflammation of the epiglottis, inflammation of the tonsils, inflammation of the oropharynx, eosinophilic esophagitis, gastroesophageal reflux disease (GERD), non-erosive reflux disease (NERD), erosive esophagitis, Barrett's esophagus, eosinophilic gastroenteritis, hypereosinophilic syndrome, corrosive (caustic) chemical esophagitis, radiation-induced esophagitis, chemotherapy-induced esophagitis, transient drug-induced esophagitis (also known as medication esophagitis), persistent drug-induced esophagitis, Crohn's disease of the esophagus, scleroderma, and scleroderma sine scleroderma, systemic lupus erythematosus, systemic vasculitides, leukocytoclastic vasculitis, polyarteritis nodosa, Churg-Strauss syndrome, rheumatoid vasculitis, and pseudomembranous esophagitis.

In other embodiments, the pharmaceutical compositions of the present invention are suitable for treating inflammatory conditions, but not limited to, an autoimmune disease, Behcet's syndrome, Kawasaki disease, X-linked lymphoproliferative syndrome; an infectious viral disease caused by one or more of the following viruses: Adenoviridae, Coronaviridae, Coxsackie virus, Herpes simplex, HIV, Influenza (Type A), Lassa virus, Epstein-Barr virus, Parainfluenza, or Respiratory syncytial virus; an infectious bacterial disease caused by one or more of the following bacteria: Arcanobacterium hemolyticum, Chlamydia (Chlamydophila), Corynebacterium, Francisella tularensis, Group A, C, G Streptococcus, S. pneumoniae, S. pyogenes, Haemophilus influenza type B, Mycoplasma pneumonia, Neisseria gonorrhea, Multiple (e.g. peritonsillar cellulitis/abscess); an infectious fungal disease caused by Candida (e.g. Candida albicans) or Histoplasma (e.g. H capsulatum); inflammation caused by injury or an irritant selected from the group consisting of an airway foreign body, chloroacetophenone, chlorobenzylidene malononitrile, chronic smoke exposure, morpholine, sulfuryl fluoride, and scalded throat; lepidopterism.

Certain embodiments of the invention include an immediate release dosage form. Additional embodiments of the invention include a controlled release dosage form. Any suitable method known in the art can be used to provide controlled release and/or delivery of the composition. In a particular embodiment of the invention, a dosage form comprising the composition and a polymer coating. Suitable polymer coatings include, but are not limited to anionic copolymers based on methacrylic acid and methyl methacrylate, such as Eudragit S, and anionic copolymers based on methyl acrylate, methyl methacrylate and methacrylic acid, such as Eugragit FS.

In certain embodiments, the dosage form may include suitable excipients.

In a specific embodiment, the following process is utilized to prepare the composition.

Young olive tree leaves are selected from the olive trees and young olive tree branches are selected and harvested at a temperature of about 25 deg C. Further, young olive green fruits are harvested at about 25 deg C. The harvested mixture is grounded at a maximum temperature of 24 deg C. Each component of the mixture is maintained at a specific percentage.

Further, the mixture is agitated and decanted at a temperature of 20 deg C. The mixture is extracted initially at cold temperature. The mixture is filtered at maximum temperature of 20 deg C. and the obtained solution is decanted and the components present in the mixture are extracted through a filter. The extracted mixture comprises polyphenols. Activity of polyphenols is maintained by controlling temperature and humidity.

The process of preparing of extraction does not use any solvents.

The present invention is further described in the exhibits that were attached as addendum to the provisional application. The exhibits are considered to be within the scope of the present disclosure and are incorporated herein in their entirety.

It should be understood that the foregoing description is only illustrative of the present inventions. Various alternatives and modifications can be devised by those skilled in the art without departing from the disclosure. Accordingly, the present disclosure is intended to embrace all such alternatives, modifications and variances. Other elements, steps, methods, and techniques that are insubstantially different from those described above and/or in the appended claims are also intended to be within the scope of the disclosure.

1. A pharmaceutical composition comprising antioxidants and phenolic compounds.

2. The pharmaceutical composition of claim 1, comprising oleuropein, hydroxytyrosol (3′4′-DHPEA) and tyrosol (p-DHPEA) and flavomoids, such as, without limiting, rutin, quercetin, luteolin and apigenin.

3. The pharmaceutical composition of claim 2 comprising caffeic acid.

4. The pharmaceutical composition of claim 2 comprising oleocanthal (p-DHPEA-EDA).

5. The pharmaceutical composition of claim 2, wherein oleuropein is present from about at least 30%, hydroxytyrosol from about at least 50%, tyrosol from at least about 25%, and oleocanthol from at least about 2%, and suitable pharmaceutical excipients.

6. An oral dosage form of the composition of claim 2.

7. The oral dosage form of claim 6 further comprising a polymer or a coating.
